(19) United States
(12) Patent Application Publication
(10) Pub. No.: US 2022/0223257 A1
(43) Pub. Date: Jul. 14, 2022

(54) GENERATION OF PERSONALIZED NEUROPROTECTIVE AND CARDIOPROTECTIVE NUTRITION PROGRAMS FEATURING CALORIC RESTRICTION

(71) Applicant: THE REGENTS OF THE UNIVERSITY OF CALIFORNIA, Oakland, CA (US)

(72) Inventors: Yama Akbari, Irvine, CA (US); Matine Azadian, Irvine, CA (US); Robert H. Wilson, Irvine, CA (US)

(21) Appl. No.: 17/706,217

(22) Filed: Mar. 28, 2022

Related U.S. Application Data
(63) Continuation-in-part of application No. PCT/US20/53144, filed on Sep. 28, 2020.
Application No. PCT/US20/53144 is a continuation-in-part of application No. 17/690,866, filed on Mar. 9, 2022.
Application No. 17/690,866 is a continuation-in-part of application No. 17/534,986, filed on Nov. 24, 2021.
Application No. 17/534,986 is a continuation-in-part of application No. PCT/US20/35440, filed on May 29, 2020.
Application No. 17/534,986 is a continuation-in-part of application No. 17/377,123, filed on Jul. 15, 2021.
Application No. 17/377,123 is a continuation-in-part of application No. 16/985,113, filed on Aug. 4, 2020, now abandoned.
Application No. 16/985,113 is a continuation-in-part of application No. 16/837,478, filed on Apr. 1, 2020, now abandoned.
Application No. 16/837,478 is a continuation-in-part of application No. PCT/US20/35440, filed on May 29, 2020.
Application No. 17/534,986 is a continuation-in-part of application No. PCT/US20/53144, filed on Sep. 28, 2020.
Application No. 17/534,986 is a continuation-in-part of application No. 17/277,616, filed on Mar. 18, 2021.
(60) Provisional application No. 62/907,595, filed on Sep. 28, 2019.
(60) Provisional application No. 62/854,215, filed on May 29, 2019.
(60) Provisional application No. 62/827,668, filed on Apr. 1, 2019.
(60) Provisional application No. 63/032,491, filed on May 29, 2020.
(60) Provisional application No. 62/854,215, filed on May 29, 2019.
(60) Provisional application No. 62/907,595, filed on Sep. 28, 2019.
(60) Provisional application No. 62/734,417, filed on Sep. 21, 2018.

(51) Int. Cl. G16H 20/60; A61B 5/00

(52) U.S. Cl. CPC G16H 20/60; A61B 5/7275; A61B 5/4866

(57) ABSTRACT

Systems and methods for generating neuroprotective and cardioprotective nutrition programs are described herein. These neuroprotective and cardioprotective nutrition programs are especially applicable for patients at risk of cardiac arrest (e.g. due to hypoxia or ischemia of the brain or other body parts). The programs may feature caloric restriction, for example, short-term caloric restriction. The programs may be generated or iteratively modified based on the hemodynamic and metabolic state of the patient's brain, limbs, or other tissues or organs. Dynamic feedback about the patient's hemodynamic and metabolic state may be provided by techniques including, but not limited to, optical technology for quantitatively and noninvasively measuring blood flow, oxygenation, metabolic rate of oxygen, and perfusion/metabolism ratio in the brain, limbs, or other tissues or organs. The systems described herein may also induce spreading depolarization and repolarization at specific times during or after cardiac arrest based on the patient's cerebral metabolic state.

### CROSS-REFERENCES TO RELATED APPLICATIONS

This application is a continuation-in-part and claims benefit of PCT Application No. PCT/US2020/053144, filed Sep. 28, 2020, which is a PCT application and claims benefit of U.S. Patent Application No. 62/907,595, filed Sep. 28, 2019, the specifications of which are incorporated herein in their entirety by reference.

This application is a continuation-in-part and claims benefit of U.S. patent application Ser. No. 17/690,866 filed Mar. 9, 2022 which is a continuation-in-part and claims benefit of U.S. patent application Ser. No. 17/534,986 filed Nov. 24, 2021, the specification of which is incorporated herein in its entirety by reference.

U.S. patent application Ser. No. 17/534,986 is a continuation-in-part and claims benefit of PCT Application No. PCT/US2020/035440 filed May 29, 2020, which claims benefit of U.S. Provisional Application No. 62/854,215, filed May 29, 2019, the specification(s) of which is/are incorporated herein in their entirety by reference.

U.S. patent application Ser. No. 17/534,986 is also a continuation-in-part and claims benefit of U.S. patent application Ser. No. 17/377,123 filed Jul. 15, 2021, which is continuation-in-part and claims benefit of U.S. patent application Ser. No. 16/985,113 filed Aug. 4, 2020, now abandoned, which is a continuation-in-part and claims benefit of U.S. patent application Ser. No. 16/837,478 filed Apr. 1, 2020, now abandoned, which is a non-provisional and claims benefit of U.S. Provisional Application No. 62/827,668 filed Apr. 1, 2019, the specification(s) of which is/are incorporated herein in their entirety by reference.

Also, U.S. patent application Ser. No. 16/985,113 is a non-provisional and claims benefit of U.S. Provisional Application No. 63/032,491 filed May 29, 2020, the specification(s) of which is/are incorporated herein in their entirety by reference.

Further, U.S. patent application Ser. No. 16/985,113 is a continuation-in-part and claims benefit of PCT Application No. PCT/US2020/035440 filed May 29, 2020, which claims benefit of U.S. Provisional Application No. 62/854,215 filed May 29, 2019, the specification(s) of which is/are incorporated herein in their entirety by reference.

U.S. patent application Ser. No. 17/534,986 is also a continuation-in-part and claims benefit of PCT Application No. PCT/US2020/053144 filed Sep. 28, 2020, which claims benefit of U.S. Provisional 62/907,595 filed Sep. 28, 2019, the specification(s) of which is/are incorporated herein in their entirety by reference.

U.S. patent application Ser. No. 17/534,986 is also a continuation-in-part and claims benefit of U.S. patent application Ser. No. 17/277,616 filed Mar. 18, 2021, which is a 371 and claims benefit of PCT Application No. PCT/US2019/052486 filed Sep. 23, 2019, which claims benefit of U.S. Provisional Application No. 62/734,417 filed Sep. 21, 2018, the specification(s) of which is/are incorporated herein in their entirety by reference.

### STATEMENT REGARDING FEDERALLY SPONSORED RESEARCH OR DEVELOPMENT

This invention was made with government support under Grant No. KL2 TR001416, UL1 TR001414, and R21 EB024793 awarded by NIH. The government has certain rights in the invention.

### BACKGROUND OF THE INVENTION

### Field of the Invention

The present invention relates to systems and methods for the generation of personalized neuroprotective or cardioprotective nutrition programs for patients at risk of cardiac arrest.

### Background Art

Nutrition programs have multiple goals. A primary goal is to allow an individual to make dietary decisions which provide the individual with sufficient nutrition in order to sustain life. More advanced nutrition programs may guide the individual towards goals of weight loss or gain, muscle development, cognitive enhancement or optimization, cardiovascular enhancement or optimization, achievement of ketosis, or the adjustment of various biometric parameters such as blood pressure. Nutrition programs may be for a short term, an indefinite term, or a term with any intermediate duration. Nutrition programs may be static over the program term or may be iteratively updated based on measured changes in the individual's condition.

Caloric restriction (CR) is defined as a reduction in caloric intake and can be daily, life-long, or intermittent. Many of the benefits appear to target aging, which includes prolongation of lifespan, and improvements in age-related deficits of learning and memory. In addition to its effects on aging, CR has been shown to be beneficial in various models of neurological diseases, like Alzheimer's, Parkinson's, and epilepsy, as well as having neuroprotective effects in various models of traumatic brain injury and stroke. Studies involving short-term overnight caloric restriction prior to global ischemic insult have not been previously investigated. Pre-clinical analysis of such phenomena can provide approaches to ameliorate recovery following focal cerebral ischemia, which on average affects over 795,000 people per year in the US alone.

Cardiac arrest (CA) afflicts over half a million individuals in the USA annually with costs to society ranging close to $50 billion per year. Survival rates have remained abysmal over the past few decades with only 5-11% of out-of-hospital and ˜17% of in-hospital CA sufferers surviving. Almost 90% of CA survivors emerge in a comatose state with severe neurological damage needing significant medical care. After cardiopulmonary resuscitation (CPR), neurologic outcome is poor because devastating hypoxic-ischemic damage to the brain has already occurred.

Predicting outcome after CA can help determine the likelihood of patients emerging from a comatose or vegetative state to an awake and responsive state. Currently, the main information that is helpful for prognostication is the type (shockable or non-shockable rhythm) and duration of CA before resuscitation efforts have begun. For example, knowing that a patient has suffered a very prolonged CA in a non-shockable state forebodes a very poor prognosis, whereas if help and resuscitation starts within seconds or <5 minutes after CA (especially a shockable rhythm), there is a meaningful chance of a good recovery if resuscitation is successful. After resuscitation, the main tools available for prognostication are neurological exams, brain imaging (e.g. CT, MRI), electrophysiologic testing (e.g. electroencephalography and somatosensory evoked potentials), and blood tests (e.g. neuron-specific enolase). However, these tests only provide value hours or days (e.g. 24-72 hours) after resuscitation and many of the processes responsible for ischemic damage and reperfusion injury in the brain are well-underway by the time these exams are completed.

Spreading depolarization (SD) is a self-propagating wave of neuronal depolarization that results in cytotoxic edema of neurons. With an inability to maintain membrane potential, SD marks near complete loss of neuronal activity in energy-compromised tissue. SD-related phenomena are seen in a multitude of conditions including migraine aura, traumatic brain injury, hypoxia, ischemia, as well as experimental manipulations such as administration of KCl directly onto the brain. Ischemia-induced SD results in the morphological alteration of neurons, including damage to dendritic spines. This “wave of death” has been shown to mark the onset of cytotoxic events (e.g., glutamate release, Ca2+ influx, cytotoxic edema), however, this damage can be reversed with timely reperfusion. Further still, SD is particularly harmful to brain parenchyma and cerebral vasculature in a hypoxic/ischemic state, like that which occurs during cardiac arrest. Without intervention, CA results in terminal spreading depolarization. Quantifying SD during CA and resuscitation may provide an important tool for diagnosis, prognosis, and possible therapeutic interventions during neurological injury during hypoxic-ischemic events. Repolarization is a corresponding self-propagating wave of restoration of neuronal membrane potential following resuscitation and return of spontaneous circulation post-CA. Repolarization marks restoration of neuronal activity within the first ˜3 min post-resuscitation, and the cerebral blood flow and brain perfusion/metabolism relationship during this time may play a crucial role in diagnosis of injury and prognosis of recovery. Therefore, quantifying repolarization post-resuscitation may provide an additional important tool for diagnosis, prognosis, and possible therapeutic intervention immediately post-CA.

### BRIEF SUMMARY OF THE INVENTION

It is an objective of the present invention to provide systems and methods for generating personalized neuroprotective or cardioprotective nutrition programs, as specified in the independent claims. Embodiments of the invention are given in the dependent claims. Embodiments of the present invention can be freely combined with each other if they are not mutually exclusive.

Caloric restriction (CR) can increase the life and health span of a broad range of species. However, the effects of overnight CR on brain functions are far from explored. Without wishing to limit the present invention to a particular theory or mechanism, the inventors have surprisingly discovered that short-term (e.g. overnight) caloric restriction (75% for 14 h) prior to cardiac arrest and resuscitation (CA) leads to an increase in survivability and improvement in neurological recovery, including reduced neurodegeneration in multiple regions of the brain. The present invention has also found that overnight CR induces normoglycemia, while significantly decreasing levels of blood glucose, insulin, and glucagon production and increasing corticosterone and ketone body production. Furthermore, the observed beneficial effects of overnight CR are independent of SIRT-1 and BDNF upregulation.

Chronic caloric restriction has been previously associated with various biological effects and purported health benefits. However, the acute or short-term effects of caloric restriction, and specifically the neuroprotective and cardioprotective effects of nutrition programs which feature short-term caloric restriction, have not been fully appreciated. Short-term caloric restriction differs from chronic caloric restriction in that shorter periods don't offer as much time to modify pathways typically affected by traditional dietary regimens of calorie restriction, which are predominantly in the chronic state. Indeed, the majority of benefits seen to date in caloric restriction are in the chronic state (whether continuous or intermittent restriction in calories) and the mechanisms often include changes in autophagy, brain-derived neurotrophic factor, or other pathways that typically require longer periods of time beyond 12-24 hours.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWINGS

The features and advantages of the present invention will become apparent from a consideration of the following detailed description presented in connection with the accompanying drawings in which:

FIG. 1 is a graph of arterial blood glucose of control vs. CR rats. Blood glucose was measured 10 minutes prior to CA and 10 minutes after resuscitation. CR significantly lowers and stabilizes blood glucose through the period of CA+CPR. **P<0.01; by one-way analysis of variance. CA, cardiac arrest; CON, control; SEM, standard error of the mean.

FIG. 2 is a graph of fraction survival of post-CA rats. No mortalities occurred in the CR group. There was a statistically significant difference in survival distributions for the CR versus control group. p<0.05 by Kaplan-Meier survival analysis. CA, cardiac arrest; CON, control.

FIG. 3 shows neurological recovery of post-CA rats. NDS was measured at 4, 24, 48, and 72 h post-CA. Rats in the CR group consistently scored significantly higher at all measured timepoints. *p<0.05 by two-way analysis of variance with post-hoc t-tests. CA, cardiac arrest; CON, control; NDS, Neurological Deficit Scale; SEM, standard error of the mean.

FIGS. 4A-B show the burst suppression ratio (BSR) of control vs. CR rats. FIG. 4A shows BSR beginning at asphyxia (time 0) until approximately 4-hrs post-CA. Statistical analysis revealed a significant group-time interaction when examining 30 minutes to the end of ECoG recording to exclude the dynamic period of asphyxia and CPR. p<0.01; by repeated measures ANOVA. FIG. 4B shows the median BSR over 5 minutes of pre-CA isoflurane (2%) anesthesia period; no statistical difference was found between the two groups. p=0.60; by Mann-Whitney U-test.

FIGS. 5A-5E show neurodegeneration in multiple brain regions at 72 h post-CA. FIG. 5A shows a brain atlas. The region within the red square displays the physiological locales of neurodegeneration as shown in FIG. 5B. FIG. 5B show brain sections stained with FJ-B; positive neurons are fluorescent (green). Images were cropped and signal intensities were adjusted linearly to be optimal for demonstration. In FIGS. 5C-5E, the number of FJ-B positive neurons were counted from 3 brain sections per region in each animal. The number of FJ-B positive neurons were significantly higher in control rats; CR rats exhibited no FJ-B positive neurons at these regions. *p<0.05 by unequal variances t-test. CA, cardiac arrest; FJ-B, Fluorojade-B, CON, control; SEM, standard error of the mean.

FIG. 6 shows capillary blood ketone ((3-hydroxybutyrate) levels of control vs. CR rats. Blood ketones were measured after 14-hrs of caloric restriction. CR significantly increases ketone levels. **P<0.01; by unpaired t-test. CR, caloric restriction; CON, control; SEM, standard error of the mean.

FIGS. 7A-7C show arterial blood corticosterone, glucagon, and insulin levels of control vs. CR rats measured after 14-hrs of caloric restriction. CR significantly increases corticosterone levels and lowers glucagon and insulin levels. *P<0.05; by Welch's t-test. CR, caloric restriction; CON, control; SEM, standard error of the mean.

FIGS. 8A-8B show BDNF and SIRT-1 in brain homogenates following 14-hrs of CR. In FIG. 8A, the Western blot was probed with SIRT1 antibody (AHP1003) and showed no difference between CR (n=3) and control groups (n=3; p=0.78). It was then stripped and reprobed with an anti-tubulin antibody to confirm loading equivalence. In FIG. 8B, the Western blot was probed first with BDNF antibody (AHP1003) and showed no significant difference between CR (n=3) and controls (n=3; p=0.16). It was then stripped and reprobed with an anti-tubulin antibody to confirm loading equivalence. Lane 1-3 are control samples, Lane 3-6 are CR samples.

FIG. 9 shows ultra-short (overnight) CR hastens the onset of the spreading depolarization.

FIG. 10 illustrates the effect of overnight CR on three rats. The cerebral perfusion/metabolism ratio CBF/CMRO₂ was measured following isoflurane washout prior to and after overnight caloric restriction. The graph shows that overnight caloric restriction led to an increased CBF/CMRO₂ ratio that became closer to 1 for each of the three rats.

FIGS. 11A-11B illustrate correlations between blood glucose levels in four wild-type aged mice imaged on the same day and both absolute CMRO₂ (μM O₂/min) and the absolute CBF/absolute CMRO₂ ratio. FIG. 11A shows a negative relationship between glucose and absolute CMRO₂. FIG. 11B shows a positive relationship between glucose and the absolute CBF/absolute CMRO₂ ratio.

FIG. 12A-12B demonstrate experimental data from representative rats that underwent CR or ad-lib diet, underwent cardiac arrest and resuscitation followed by euthanasia at 72 hours post-resuscitation. To assess for cardioprotection after ultra-short CR, which supports the data in FIG. 2 above, rat's hearts were isolated from both groups and histological staining was conducted to assess for early changes in cardiomyocytes. On Masson trichrome stain, lower amounts of myocardial scar tissue were found in CR rats. This is an established marker of cardioprotection from ischemic insults. FIG. 12A shows heart sections of CR and non-CR rats at 72 hrs post-CA stained with Masson trichrome (red: cytoplasm and muscle fibers; purple: collagen; black: nuclei). There are more collagen-rich myocardial scar tissue (arrow) and focal islands of collagen deposition (arrow head) in walls of non-CR left ventricle versus in the walls of CR left ventricle. Furthermore, vanadium acid fuchsin (VAF) stain, an established marker of cellular ischemic damage, was conducted and demonstrated that CR reduces the amount of VAF-stained cardiomyocytes, further supporting CR-mediated cardioprotection. FIG. 12B shows heart sections of CR and non-CR rats at 72 hrs post-CA that were stained with vanadium acid fuchsin (purple) and toluidine (blue). There are more purple cardiac myocytes (arrow) in non-CR hearts versus CR hearts, indicating that non-CR cardiac myocytes are more susceptible to ischemic damage

### DETAILED DESCRIPTION OF THE INVENTION

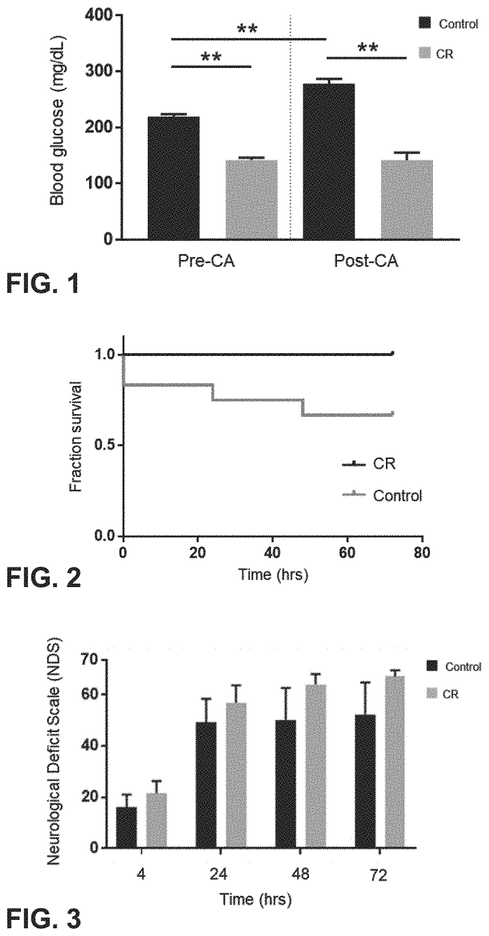

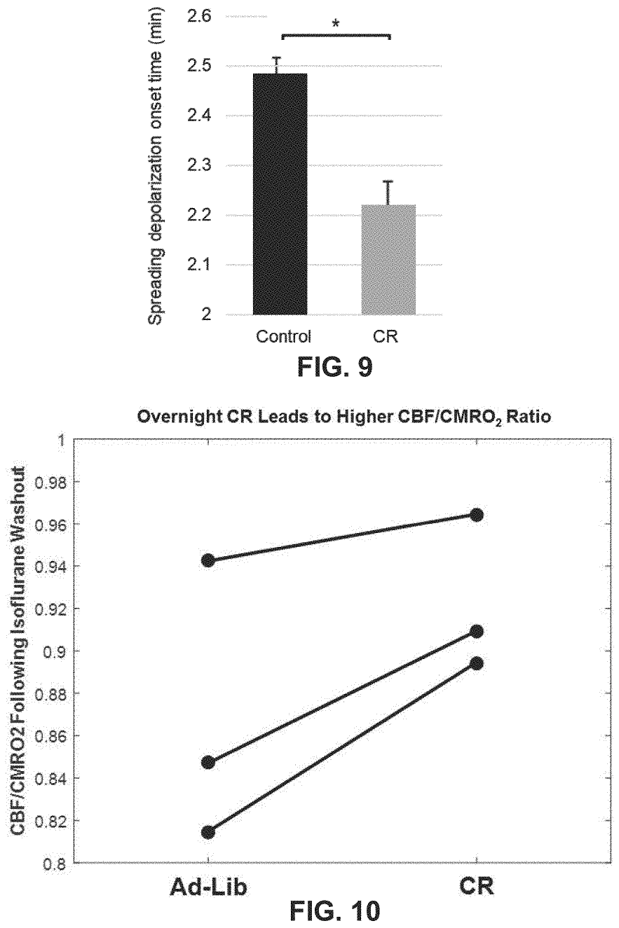

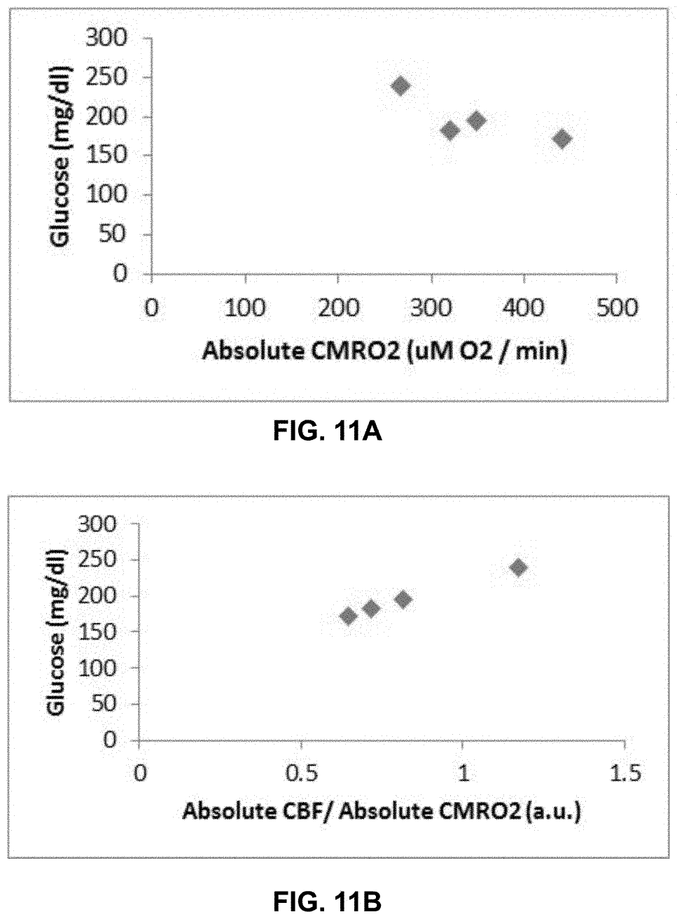

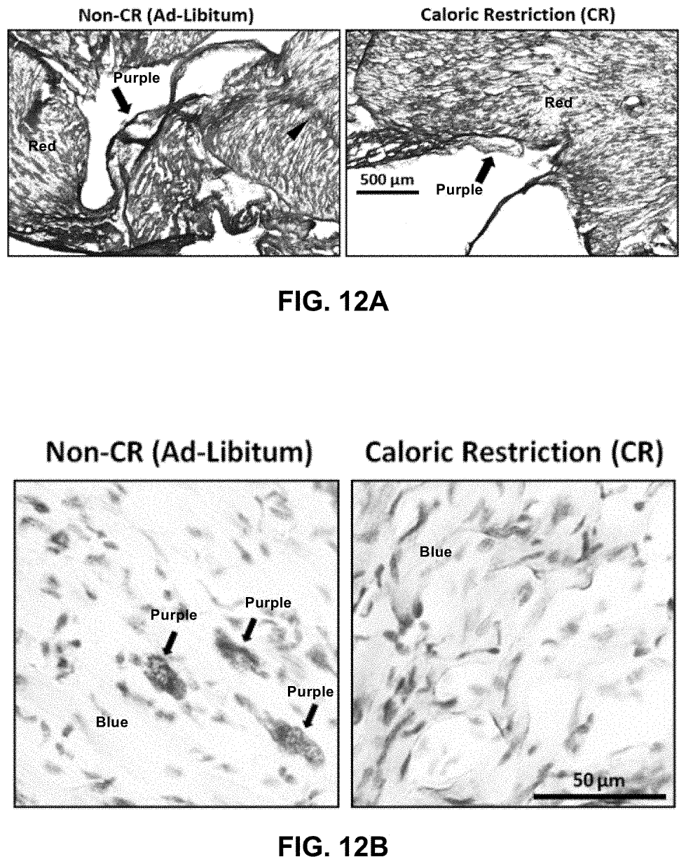

Referring now to FIGS. 1-12B, in some embodiments, the present invention features a system for generating a neuroprotective or cardioprotective nutrition program for a patient at-risk of cardiac arrest. This risk of cardiac arrest may be cardiac or non-cardiac in origin (e.g. may stem from a risk of hypoxia or ischemia of the brain or another part of the body). As a non-limiting example, the neuroprotective or cardioprotective nutrition program may include caloric restrictions for the patient. The neuroprotective or cardioprotective nutrition program may comprise reducing the patient's caloric intake by at least 50%. For example, the caloric intake is decreased by about 50-80%. In some embodiments, the caloric restrictions may be imposed for a period of about 10-20 hours per day. For instance, a patient that is hospitalized and at-risk of having a cardiac event may be calorically restricted for 15 hours per day by not eating during this period, or receiving only 25% of the normal amount of calories based on their age and weight (caloric restriction of 75%). As another example, a patient may be only allowed to eat for a period of 3-5 hours in the afternoon and/or evening, and possibly only a small snack for the remainder of the day.

In one embodiment, the system for generating a neuroprotective or cardioprotective nutrition program may include: a means for evaluating the patient's risk of cardiac arrest; and a means for evaluating the patient's metabolic state to determine the neuroprotective or cardioprotective potential of caloric restriction of the patient. As described herein, combination of the patient's risk of cardiac arrest and neuroprotective or cardioprotective potential of caloric restriction may allow for generation of a patient-specific neuroprotective or cardioprotective nutrition program. The means for evaluating the metabolic state of the patient may be configured to evaluate a cerebral metabolic state, a non-cerebral metabolic state, or a combination thereof. As used herein, the term “non-cerebral metabolic state” refers to a metabolic state that is measured in a portion of the body other than the brain. As a non-limiting example, a non-cerebral metabolic state may be evaluated via measurement of a peripheral limb, another tissue or organ (such as the heart or lungs), or multiple portions of the patient's body. In one embodiment, the system may be configured to allow for comparison of a cerebral metabolic state and a non-cerebral metabolic state so as to assess autoregulation.

The system may use any of a variety of means to evaluate the patient's risk of cardiac arrest. In some embodiments, it may be determined that the patient is at risk of cardiac arrest because they have already had a cardiac event, either recently or some time ago. In that case, the means to evaluate the patient's risk of cardiac arrest may be the patient's medical history records.

Similarly, the system may use any of a variety of means to evaluate the patient's metabolic state. This evaluation may focus on a comprehensive metabolic state of the entire body, a local metabolic state (such as a cerebral metabolic state or the metabolic state of another tissue), or a relationship between the metabolism (or flow/metabolism ratio) of the brain and that of other tissues (i.e. an autoregulation ratio). As a non-limiting example, the system may use optical devices to measure cerebral blood flow and oxygenation (e.g., laser Doppler flowmetry or diffuse correlation spectroscopy to measure blood flow and near-infrared spectroscopy, time-resolved spectroscopy, or frequency-domain spectroscopy to measure brain tissue concentrations of oxygenated and deoxygenated hemoglobin. These data may then be combined to obtain metrics including, but not limited to, absolute or relative cerebral metabolic rate of oxygen (CMRO₂) and flow-metabolism ratio (CBF/CMRO₂). The cerebral metabolic state of the patient may be used to determine the neuroprotective or cardioprotective potential of caloric restriction of the patient. A threshold may be placed on the value or values of one or more of these metrics to distinguish a patient for which caloric restriction is expected to have significant neuroprotective or cardioprotective effects from a patient for which caloric restriction is not expected to provide notable neuroprotection or cardioprotection. Alternatively, one or more of these metrics may be treated as continuous independent variables in a mathematical model (e.g., a linear regression) to predict the amount of caloric restriction (e.g., 25%, 50%, or 75% of baseline caloric intake) recommended for the patient to have a significant chance of obtaining neuroprotection or cardioprotection.

The following are non-limiting examples of how the combination of the patient's risk of hypoxia or ischemia to the brain or other body parts that may lead to a cessation of brain or bodily blood flow and metabolism, (e.g. cardiac arrest) and the neuroprotective or cardioprotective potential of caloric restriction may allow for generation of the neuroprotective nutrition program. In some embodiment the combination of the patient's risk of cardiac arrest and the neuroprotective or cardioprotective potential of caloric restriction may allow for generation of the neuroprotective or cardioprotective nutrition program. As a non-limiting example, this decision can be guided by training a multivariate linear regression of the form y=k₁x₁+k₂x₂ where “y” represents the percentage of caloric intake to restrict, “x₁” is the patient's relative risk of having cardiac arrest during the hospital stay, “x₂” is a metabolism variable related to oxygen and glucose supply and cerebral metabolic rate of oxygen, and k₁ and k₂ are the weighting coefficients of x₁ and x₂, respectively. This algorithm can be trained (using an initial data set) to obtain the values of k₁ and k₂. Once the algorithm is trained, k₁ and k₂ will be known, so any subsequent patient with measurable values of x₁ and x₂ can be calorically restricted by “y”%, where “y” is calculated separately for each new patient using the regression model.

In one embodiment, the combination of data on oxygen or glucose supply and metabolic rate may allow for evaluation of the neuroprotective or cardioprotective potential of caloric restriction of the patient. As a non-limiting example, the data can be represented by a coordinate in a multi-dimensional space (e.g., the coordinate (O₂, Glu, CMRO₂) in a 3-dimensional space, where the x-axis represents oxygen supply, the y-axis represents glucose supply, and the z-axis represents cerebral metabolic rate of oxygen). Each patient's (O₂, Glu, CMRO₂) coordinate will be compared with previously defined thresholds in this 3-D space (represented as 2-D planes) to classify that individual patient as having “high potential”, “moderate potential”, and “low potential” to gain neuroprotection or cardioprotection from caloric restriction.

In some embodiments, the present invention features a neuroprotective or cardioprotective nutrition program for a patient. The neuroprotective or cardioprotective nutrition program may include a neuroprotective dietary limit. As a non-limiting example, the neuroprotective or cardioprotective dietary limit may be a caloric limit or may exclude or limit certain foods or food groups. In some embodiments, the neuroprotective or cardioprotective nutrition program may include a set ratio of macronutrients such as carbohydrates, fats, proteins, sugars, or other energy-providing nutrients such as ketone bodies or nutrients that feed into the anaerobic glycolysis and/or aerobic citric acid cycle. As a further non-limiting example, the caloric limit may cap consumption at about 50, 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400 or 3500 kilocalories per half-hour, hour, meal, morning, afternoon, 12-hour period, 24-hour period, 36-hour period, 48-hour period, or 72-hour period. In some embodiments, the neuroprotective or cardioprotective nutrition program may be for a period of less than 72 hours. In other embodiments, the neuroprotective or cardioprotective nutrition program may be for a period of less than 1, 2, 3, 4, 5, 6, 7, 8, 9, 10, 11, 12, 13, 14, 15, 16, 17, 18, 19, 20, 21, 22, 23, 24, 26, 28, 30, 32, 34, 36, 40, 44, 48, 60, 70, 80, 90 or 100 hours, or may be for a period of 1, 2, 3, 4, 5, 6, 7, 8, 9, 10, 11, 12 or more days, weeks, or months.

Non-limiting examples of means for evaluating the patient's risk of hypoxia and/or ischemia (e.g. cardiac arrest) to the brain and body include means for evaluating clinical signs and symptoms (e.g. chest pain), an oxygen saturation monitor (e.g. pulse oximetry), a blood pressure monitor, a heart rate monitor, a heart rhythm monitor (e.g. an electrocardiogram device), a serum laboratory profile (e.g. cardiac enzymes, blood sugar, inflammatory markers, lactate, renal function labs, coagulopathy labs, platelet function assays, etc), a coronary catherization or angiogram device, a chest x-ray device, an echocardiogram device, a computed tomography (CT) device, a magnetic resonance imaging (MRI) device, a diffuse optical spectroscopy device, a near infrared spectroscopy (NIRS) device, a perfusion monitoring device, a laser Doppler flowmetry device, a nuclear scan device, a genetic test, or a combination thereof. Non-limiting examples of means for evaluating a metabolic state of the patient include: an optical hemodynamic monitor, a blood sugar test using a rapid handheld glucometer, or an indicator of ketosis level, the latter of which can be tested by serum or urine samples including with rapid bedside testing. In some embodiments, the means for evaluating a metabolic state of the patient may be configured to measure absolute or relative values of one or more of the following parameters: cerebral metabolic rate of oxygen (CMRO2), cerebral metabolic rate of glucose, cerebral blood flow (CBF), flow-metabolism ratio (CBF/CMRO2) tissue concentration of deoxy-hemoglobin (ctHb), tissue concentration of oxygenated hemoglobin (ctHbO2), tissue oxygenation (StO2), tissue water content, tissue lipid content, tissue reduced scattering coefficient, tissue scattering amplitude, tissue scattering slope, tissue reflectance, or a combination thereof.

As additional non-limiting examples, the patient's cerebral or non-cerebral metabolic state may be evaluated based on most recent meal times, corticosterone levels, glucagon levels, insulin levels, ketone levels, or a combination thereof. In some embodiments the means for evaluating the patient's cerebral or non-cerebral metabolic state may measure one or more analytes from a biological sample (such as blood, tissue, saliva, sweat, cerebral spinal fluid, respiratory gas sample, or a urine sample) taken from the patient. In other embodiments the means for evaluating the patient's cerebral or non-cerebral metabolic state may be a wearable device. This wearable device may consist of a miniaturized light source and detector and associated optics (e.g. MEMS/MOEMS technology) built into a patch or elastic band that attaches to the head, limb, or another part of the body. This device may detect the backscattered light at one or more wavelengths and modulation frequencies to obtain tissue optical properties such as absolute or relative blood flow, hemoglobin concentration, oxygenation, and cerebral metabolic rate of oxygen (or ratios of these parameters). If any of these properties (or a combination thereof) crosses a certain threshold, an indicator on the wearable device (e.g., a light or an alarm) may be activated to alert the patient and clinicians about this hemodynamic change. This technology may be used as a feedback mechanism to inform a personalized caloric restriction program to optimize neuroprotection or cardioprotection for each patient.

In one embodiment, the means for evaluating the patient's metabolic state features a system for quantitative intracranial measurement of cerebral blood flow, oxygenation, metabolism, autoregulation, or a combination thereof. As a non-limiting example, the system for evaluating the patient's metabolic state may comprise: a device body; one or more light sources; one or more detectors, a microprocessor, and a memory component. The light sources and the detectors may extend from the device body and be configured to be positioned in proximity to a head of a subject. As a non-limiting example, the light sources and the detectors may extend from the device body so as to pass through the hair of the subject and contact the skin surface at a plurality of points in a measurement area. In another embodiment, the light sources and detectors may not extend from the device body, but instead be integrated within an end of the device body. In some embodiments, the microprocessor may be operatively connected to the one or more light sources, the one or more detectors, or a combination thereof. In further embodiments, the memory component may be operatively connected to the microprocessor, and the microprocessor may be capable of executing instructions held or stored in the memory component. According to preferred embodiments, one or more of the light sources may be configured to emit a coherent light signal. In selected embodiments, the system may be configured to detect and decouple one or more backscattered signals via the detectors. In one embodiment, the memory component may comprise instructions for decoupling components of the one or more backscattered signals. As a non-limiting example, the system may be configured to: differentiate between components of the one or more backscattered signals which are due to different layers of the head; measure or determine a dynamic perfusion metric; measure or determine a tissue absorption coefficient; measure or determine a tissue reduced scattering coefficient; calculate a value of an absolute perfusion metric, using the dynamic perfusion metric, the tissue absorption coefficient, and the tissue reduced scattering coefficient; calculate a value of an absolute metabolic metric, using the absolute perfusion metric, the tissue absorption coefficient, and the tissue reduced scattering coefficient; and calculate a quantitative value of cerebral autoregulation, using the absolute values of the perfusion metric and the metabolic metric. As another non-limiting example, the instructions for decoupling components of the one or more backscattered signals may comprise differentiating between components of the one or more backscattered signals that are due to different layers of the head; determining a dynamic perfusion metric using the one or more backscattered signals; determining a tissue absorption coefficient using the one or more backscattered signals; determining a tissue reduced scattering coefficient using the one or more backscattered signals; calculating a value of an absolute perfusion metric, using the dynamic perfusion metric, the tissue absorption coefficient, and the tissue reduced scattering coefficient; calculating a value of an absolute metabolic metric, using the absolute perfusion metric, the tissue absorption coefficient, and the tissue reduced scattering coefficient; calculating a quantitative value of cerebral autoregulation, using the absolute values of the perfusion metric and the metabolic metric; or a combination thereof, thereby providing for quantitative intracranial measurement of cerebral blood flow, oxygenation, metabolism, and autoregulation As used herein, the terms “tissue absorption coefficient” and “tissue reduced scattering coefficient” may have both hemodynamic and non-hemodynamic components, and they may have tissue-based (e.g., brain, skin, muscle, heart, lung, etc.) and non-tissue-based (e.g., implantable or injectable tissue sensors such as implantable probes, nanoparticles, quantum dots, etc.) components.

In some embodiments the present invention may feature a system for evaluating the neuroprotective or cardioprotective potential of caloric restriction of a patient. As a non-limiting example, the system may include: a means for evaluating oxygen or glucose supply to the patient's brain; and a means for evaluating the patient's cerebral metabolic rate of oxygen or glucose. According to preferred embodiments, the combination of data on oxygen or glucose supply and cerebral metabolic rate may allow for evaluation of the neuroprotective or cardioprotective potential of caloric restriction of the patient. For example, a high neuroprotective potential of caloric restriction would indicate that the patient would have a large potential for caloric restriction to have a neuroprotective effect and a low neuroprotective potential of caloric restriction would indicate that the patient would have a low potential for caloric restriction to have a neuroprotective effect. The system may additionally include a means for evaluating a non-cerebral metabolic rate of oxygen or glucose. Comparison of the cerebral and non-cerebral metabolic rates of oxygen or glucose may allow for an assessment of the patient's state of autoregulation. A threshold may be placed on one or more parameters relating these different metabolic rates (e.g., the ratio of cerebral metabolic rate of oxygen to non-cerebral metabolic rate of oxygen) to determine whether a patient is expected to receive significant neuroprotective or cardioprotective effects from caloric restriction. One or more parameters relating these metabolic rates may also be treated as a continuous independent variable in a mathematical model (e.g., a linear regression model) to predict the optimal amount of caloric restriction (e.g., 25%, 50%, or 75% of baseline caloric intake) recommended for a specific patient to have a significant chance of obtaining neuroprotection or cardioprotection.

In one embodiment, the present invention features a method for generating a neuroprotective or cardioprotective nutrition program for a patient. As a non-limiting example, the method may comprise: evaluating the patient's risk of cardiac arrest; evaluating the patient's cerebral or non-cerebral metabolic state; and generating a neuroprotective or cardioprotective nutrition program for the patient based on combination of the patient's risk of cardiac arrest and cerebral or non-cerebral metabolic state. In some embodiments, the method may comprise re-evaluating the patient's cerebral or non-cerebral metabolic state after a time period and amending the neuroprotective or cardioprotective nutrition program based on a change in the cerebral or non-cerebral metabolic state, for example, in an iterative manner.

In another embodiment, the present invention features a system for improving neurological outcome in a patient during or after cardiac arrest. As a non-limiting example, the system may comprise: a means for evaluating the patient's metabolic state (e.g. cerebral or non-cerebral metabolic state); and a means for inducing spreading depolarization of the patient. In a preferred embodiment, the system may allow for inducement of spreading depolarization of the patient at a specific time during or after cardiac arrest, based on the patient's cerebral or non-cerebral metabolic state. Non-limiting examples of means for inducing spreading depolarization include: a therapeutic configured to place the patient in a state that mimics a caloric restricted state, a therapeutic treatment configured to increase ketone levels, a therapeutic treatment configured to lower glucagon and insulin levels, chemical stimulation, physical stimulation, electrical stimulation, magnetic stimulation, optical stimulation, soundwave-induced stimulation, or a combination thereof. In another non-limiting example, the means for inducing spreading depolarization may comprise imposing caloric restrictions on the patient prior to the cardiac arrest.

According to some embodiments, the system may be configured to induce spreading depolarization at an earlier time or a later time depending on the patient's cerebral or non-cerebral metabolic state. The system may additionally comprise a means for inducing repolarization in the patient at a specific time after reperfusion, re-oxygenation, or resuscitation. In some embodiments, this time may depend on the metabolic state of the patient. As a non-limiting example, the means for inducing repolarization may be configured to modify the patient's blood pressure, cerebral blood flow, cerebral metabolism, or a combination thereof, via chemical stimulation, physical stimulation, electrical stimulation, magnetic stimulation, optical stimulation, soundwave-induced stimulation, or a combination thereof.

In an embodiment, the present invention features a system for evaluating the neuroprotective or cardioprotective potential of caloric restriction of a patient. As a non-limiting example, the system may comprise: a means for evaluating the patient's risk of cardiac arrest; and a means for evaluating the patient's cerebral or non-cerebral metabolic state. The combination of the patient's risk of cardiac arrest and cerebral or non-cerebral metabolic state may allow for evaluation of the neuroprotective or cardioprotective potential of caloric restriction of the patient.

In another embodiment, the present invention features a system for guiding neuroprotective or cardioprotective caloric restriction of a patient. As a non-limiting example, the system may comprise: a means for evaluating the patient's risk of cardiac arrest; and a means for iteratively evaluating the patient's metabolic state (e.g. cerebral or non-cerebral metabolic state). Combination of the patient's risk of cardiac arrest and metabolic state may allow for guided neuroprotective or cardioprotective caloric restriction of the patient. As a non-limiting example, the effects (e.g. neuroprotective or cardioprotective effects) of caloric restriction of a patient may be continuously or iteratively monitored via evaluation of the patient's metabolic state, and the program of caloric restriction may be adjusted accordingly so as to optimize health and provide for neuroprotection or cardioprotection of the patient.

In yet another embodiment, the present invention features a method of determining a neuroprotective or cardioprotective dietary limit for a patient. As a non-limiting example, the method may comprise: evaluating the patient's risk of cardiac arrest; evaluating the patient's cerebral or non-cerebral metabolic state; and determining a neuroprotective or cardioprotective dietary limit for the patient based on combination of the patient's risk of cardiac arrest and metabolic state.

In still another embodiment, the present invention features a method of acute caloric restriction for neurological protection of a patient. As a non-limiting example, the method may include: determining that the patient is at risk of cardiac arrest; determining that caloric restriction of the patient has a neuroprotective or cardioprotective potential; and imposing caloric restrictions on the patient for a period of time. As a non-limiting example, the period of caloric restriction may be less than 1, 2, 3, 4, 5, 6, 7, 8, 9, 10, 11, 12, 13, 14, 15, 16, 17, 18, 20, 22, 24, 28, 32, 36, 40, 44, 48, 60 or 72 hours. In preferred embodiments, the caloric restrictions may provide for neurological protection of the patient.

In one embodiment, the present invention features a method of guiding caloric intake for neurological protection of a patient. As a non-limiting example, the method may comprise: evaluating the patient's cerebral or non-cerebral metabolic state; determining that the patient is at risk of neurological damage due to cardiac arrest, based on the metabolic state; and imposing caloric restrictions on the patient. In preferred embodiments, the caloric restrictions provide for neurological protection of the patient. In another embodiment, the present invention features a method of improving survival and neurological outcome in a patient at-risk of cardiac arrest, comprising imposing caloric restrictions on the patient for a period of less than about 72 hours. The caloric restrictions may be based on a value or a change in value of the one or more metabolic metrics. These metabolic metrics may be measured via an optical probe.

In some embodiments, the caloric restrictions of the present invention may comprise reducing the patient's caloric intake by at least 5, 10, 15, 20, 25, 30, 35, 40, 45 50, 55, 60, 65, 70, 75, 80, 85, 90 or more percent for a period of hours, days, weeks, months, or years. The caloric restrictions may necessitate the use of a nutrition program to ensure that the patient receives adequate nutrition while following a caloric restriction regime and staying within a caloric limit. In some embodiments, the caloric restrictions may be imposed for a period of about 10-20 hours per day. The caloric restrictions may also include specific changes in the nutrients to optimize potential neuroprotective or cardioprotective features in the diet, including but not limited to specific nutrients that promote a state of ketosis. Overall, the nutrition may be consumed by mouth if able to eat or by tube ending in the stomach or intestines (as needed by patients unable to eat) or intravenously (as hospitalized patients frequently need).

According to another embodiment, the invention may feature a method of improving survival and neurological outcome in a patient at-risk of cardiac arrest. In another embodiment the invention may feature a method of improving neurological survival and outcome in a patient during hypoxia or ischemia to the brain or other body parts. As a non-limiting example, the method may comprise administering to the patient a therapeutic treatment that places the patient in a state that mimics a caloric restricted state. This method may also be used during cardiac arrest. In some embodiments, the therapeutic treatment may comprise administering ketones to increase ketone levels. In other embodiments, the therapeutic treatment can lower glucagon and insulin levels.

In some other embodiments, the present invention features a method of improving survival and neurological outcome in a patient during cardiac arrest. In one aspect, the method may comprise inducing ketosis in said patient. In another aspect, the method may comprise administering to the patient a therapeutic treatment that increases ketone levels. For example, the patient is administered ketones immediately after cardiac arrest, or ketones may be administered in patients at high risk of cardiac arrest. Alternatively, or in conjunction, the method may comprise administering to the patient a therapeutic treatment that lowers glucagon and insulin levels.

Without wishing to limit the present invention to a particular theory or mechanism, the methods described herein can induce early onset spreading depolarization in the patient during cardiac arrest. In some embodiments, the methods described herein can also induce early repolarization in the patient being resuscitated from cardiac arrest.

According to some embodiments, a method of inducing early onset spreading depolarization in a patient during cardiac arrest may comprise imposing caloric restrictions on the patient or administering treatment that mimics a caloric restricted state prior to the cardiac arrest. Without wishing to limit the present invention to a particular theory or mechanism, imposing caloric restrictions or mimicking a caloric restricted state may increase ketone levels and/or lower glucagon and insulin levels.

In other embodiments, the method of inducing early onset spreading depolarization in a patient during cardiac arrest may comprise inducing ketosis in said patient. Ketosis may be induced by administering to the patient a therapeutic treatment that increases ketone levels. In conjunction with or alternative to imposing caloric restrictions and/or inducing ketosis, the method may comprise administering to the patient a therapeutic treatment that lowers glucagon and insulin levels.

In some embodiments, a method of inducing early repolarization in a patient being resuscitated from cardiac arrest may comprise administering to the patient a therapeutic treatment that increases ketone levels and/or a therapeutic treatment that lowers glucagon and insulin levels. The treatment may be administered during or immediately after cardiac arrest or during or immediately after resuscitation. For example, a patient may be injected with ketones during CPR, which is performed after spreading depolarization has taken place but before spreading repolarization is about to take place, to induce earlier spreading repolarization.

In additional embodiments, the methods described herein may further comprise inducing spreading depolarization in the patient during or immediately after cardiac arrest and/or inducing repolarization in the patient during or immediately after resuscitation by some other means. For example, spreading depolarization and/or repolarization may be induced by chemical stimulation, physical stimulation, electrical stimulation, magnetic stimulation, optical stimulation, soundwave-induced stimulation, or a combination thereof.

In some other embodiments, the present invention provides a method of prognosticating neurological outcome in a patient after cardiac arrest. The method may comprise receiving metabolic information and determining and measuring a metabolic state from said metabolic information. The metabolic state may indicative of the severity and prognosticate outcome of the neurological condition of the brain. In some embodiments, the metabolic information may comprise the most recent meal times prior to cardiac arrest, corticosterone levels, glucagon and insulin levels, and/or ketone levels. Preferably, the levels are measured from a biological sample, such as blood, tissue, saliva, sweat, cerebral spinal fluid, respiratory gas sample, or urine, taken during or after cardiac arrest. In further embodiments, the prognostication method may include detecting a presence, lack or delay of spreading depolarization and repolarization.

According to other embodiments, the present invention features a method of improving survival and neurological outcome in a patient during cardiac arrest. The method may comprise determining a metabolic state of the patient, where the metabolic state indicates severity of a neurological condition of the patient, and depending on the metabolic state, inducing spreading depolarization at a specific time during or after cardiac arrest. In one embodiment, the step of determining a metabolic state of the patient includes receiving metabolic information on the patient. The metabolic information may comprise one or more of most recent meal times prior to cardiac arrest, corticosterone levels, glucagon and insulin levels, and ketone levels. The levels may be measured from a biological sample taken during or after cardiac arrest.

In some embodiments, the patient may be in a fasted or non-fasted metabolic state. Depending on the metabolic state of the patient, spreading depolarization may be induced at an earlier time or a later time after cardiac arrest. In other embodiments, the method may further include inducing repolarization in the patient at a specific time after resuscitation, where said time depends on the metabolic state of the patient. The step of inducing spreading depolarization and/or repolarization may comprise administering to the patient a therapeutic treatment that places the patient in a state that mimics a caloric restricted state, administering to the patient a therapeutic treatment that increases ketone levels, administering to the patient a therapeutic treatment that lowers glucagon and insulin levels, chemical stimulation, physical stimulation, electrical stimulation, magnetic stimulation, optical stimulation, soundwave-induced stimulation, or a combination thereof.

### EXAMPLES

The following are non-limiting examples of the present invention. It is to be understood that said examples are not intended to limit the present invention in any way. Equivalents or substitutes are within the scope of the present invention.

### Example 1: Effects of Caloric Restriction in Rat Model

In this experiment, global ischemia by CA was induced in rats that were calorically restricted (75%) overnight for 14 h and assessed for changes in outcome. In an attempt to better understand such changes, levels of glucose, insulin, glucagon, corticosterone, and ketone bodies were measured in the blood, in addition to SIRT-1 and BDNF expression in the brain.

Materials and Methods

Animal Preparation

Adult male Wistar rats weighing 300-370 g were used. The animals were housed under standard conditions (23±2° C., 60-70% relative humidity, 12 h light and dark cycles; free access to food and water). Animals typically arrived 2 weeks prior to experiments and were handled daily for 5 min to promote habituation and reduction of stress levels. To enable monitoring of electrocorticography (ECoG), one week prior to the experiment, each rat had two electrodes (1.57 mm in diameter) implanted on the dura (2 mm anterior and 2.5 mm lateral to bregma), corresponding to the left and right M1 motor cortices of the frontal lobes. Two additional electrodes were implanted (5.5 mm posterior and 4 mm lateral to bregma), corresponding to the left and right V1 visual cortices. A reference electrode was also placed 3 mm posterior to lambda over the cerebellum. All animal procedures were approved by the University of California Animal Care Committee and conformed to the recommendations of the American Veterinary Medical Association Panel on Euthanasia.

Dietary Restriction

Rats were divided into two groups, control (CON, n=14) and calorically restricted (CR; n=14). The control group had unlimited access to food during the entire experiment. Rats from the CR group were fed 25% of the average daily food intake of the CON group. Average daily food intake was calculated as weight of standard laboratory chow pellets consumed per day per rat. CR rats were calorically restricted overnight, starting at 6:00 pm, approximately 14-hrs prior to surgical procedures at 8:00 am on the following morning and 18-hrs prior to cardiac arrest at 12:00 pm. Capillary blood ketone (β-hydroxybutyrate) levels were measured the morning after caloric restriction prior to surgical procedures with a blood glucose and ketone monitoring system. Rats in the CR group were allowed to resume ad libitum feeding during the recovery period following surgical and cardiac arrest procedures. Both groups had ad libitum access to water.

Cardiac Arrest Experiment

On the day of CA, rats were endotracheally intubated, connected to a mechanical ventilator, and maintained under 2% isoflurane anesthesia carried by 50% O₂ and 50% N₂ gas during the surgical preparations leading up to CA. The femoral artery and vein were cannulated to monitor blood pressure and heart rate and to allow for the intravenous (i.v.) administration of medications. While under mechanical ventilation, positive end expiratory pressure was maintained at 3 cmH₂O and body temperature was monitored with a rectal probe and maintained at 37° C. CA was induced via an 8-minute duration of controlled asphyxia followed by cardiopulmonary resuscitation (CPR) until return of spontaneous circulation (ROSC). No isoflurane anesthesia was administered during or after CPR for the remainder of the experiment. Approximately 250 μL of arterial blood was collected 10 minutes before asphyxia and 10 minutes after ROSC. Blood gas levels, in addition to blood glucose, were measured at both timepoints with a handhold blood analyzer. Vessels were decannulated, and when spontaneous respirations were adequate, rats were extubated.

Post-Cardiac Arrest Care

Normal saline (5 mL) and Ringer's lactate (5 mL) was administered subcutaneously (s.c.) 5 hours after CPR to limit dehydration until the rats resumed water consumption independently. Prophylactic cefazolin (45 mg/kg) was administered to limit any risk of infection. One-fourth cup of a water gel and 10 pellets of standard laboratory chow was soaked in water and placed near the rats' mouth and throughout the cage until they recovered and resumed independent consumption of standard chow. At 24 h post-CA, capillary blood ketone levels were again measured. Rats were re-examined every 24 h thereafter to ensure proper hydration and food consumption. Both CR and CON groups underwent analogous surgical, cardiac arrest, and post-cardiac arrest procedures. All rats were permitted ad libitum access to food and water following cardiac arrest.

Neurological Evaluation

Neurological recovery was quantified using the Neurological Deficit Scale (NDS; Table 1). The NDS consists of components that measure arousal, brainstem function, motor and sensory activities. Neurological recovery was evaluated at 4, 24, 48 and 72 h post-ROSC by well-trained personnel who were blind to dietary groupings.

TABLE 1
NDS for Rats
Neurological Deficit Scale (NDS) for Rats
Arousal (0-9)

| | |
|---|---|
| Alerting | Normal (10); Siuporous (5); Comatose (0); |
| Eye Opening | Open spontaneous (3); Open to pain (1) |
| Spontaneous | Normal (6); Abnormal (3); Absent (0) |
| Respiration | |

Brainstem Function (0-21)

| For each category: Normal (3); Abnormal (3); Absent (0) | |
|---|---|
| Olfaction; Vision; Pupillary Light Reflection; Corneal | |
| Reflex; Startle Reflex; Whisker Stimulation; Swallowing | |
| Motor Assessment (0-6) | |
| Strength | Normal (3); Weak (1); No movement (0) |

Sensory Assessment (0-6)

| | |
|---|---|
| Pairs | Brisk (3); Weak (1); No movement (0) |

Motor Behavior (0-6)

For each category: Normal (3); Abnormal (1); Absent (0)
Gait Coordination; Balance Beam Walking
Behavior (0-12)
For each category Normal (3); Abnormal (1); Absent (0)
Right Reflex; Visual Placing; Negative Geotaxis; Turning Alley
Best Outcome = 70; Worst Outcome = 0

Brain Tissue Collection

At 72 h following CA, survived rats were anesthetized with sodium pentobarbital and perfused transcardially with 0.9% NaCl solution, followed by 0.1 M phosphate buffered saline, pH 7.4. Brains were separated at the mid-sagittal plane into left and right hemispheres. The left hemisphere was post-fixed in 4% PFA for 24 hrs at 4° C. and cryoprotected in 30% sucrose for 4 days. It was then frozen in optimal cutting temperature (OCT) embedding medium and stored in −80° C. until sectioned. The right hemisphere was flash frozen in dry ice and stored in −80° C. until homogenization for western blot analysis

Histologic Analysis

Left brain hemispheres frozen in OCT were coronally sectioned at 30 μm using a cryostat (Microtome HM 505N). Sections were stored in serial order in a 96-well plate in 1×PBS with sodium azide at 4° C. Fluorojade-B staining was used to scan and identify neuronal degeneration at 72 h post-CA. To conform to the stereological standards of systematic random sampling (West), sections from FIG. 10 (bregma, 3.24 mm) to FIG. 155 (bregma, −14.64 mm) in the rat brain atlas (The Rat Brain in Stereotaxic Coordinates, 6^{th} edition) were chosen at a 330 μm interval (one of every twelve sections) covering a total 17880 μm scanned area of potential neurodegeneration. Selected sections were stained for 20 minutes in 0.0004% Fluorojade B solution after 10 minutes incubation with 0.06% potassium permanganate. Sections were screened under the microscope and those with Fluorojade-B positive neurons were marked for cell counting. To further avoid biased sampling, two additional sections were chosen at −90 μm anterior and +90 μm posterior of the original marked section.

Cell Counting

Images of sections were obtained on a microscope under standardized conditions, including exposure time, gain, and resolution. Square sampling fields were numerated and placed on images to encompass areas with Fluorojade-B positive neurons. A random integer generator was utilized to select half of the numerated sampling fields for cell counting. Unbiased counting frames, modified from West, were used to manually count cells, in which Fluorojade-B positive neurons that were partially or entirely within the top and right borders and did not intercept the bottom or left borders were considered to be in the counting frame and counted. This method ensured that Fluorojade-B positive neurons, regardless of size, shape, and orientation were counted never more than once. All images were blindly analyzed by three trained personnel using ImageJ Plugin “Cell Counter”.

Measurement of Blood Serum Analytes

Arterial blood samples collected during the CA experiment were processed in EDTA-coated tubes with 25 μL aprotinin. After centrifugation (1,000×g, 15 min), serum samples were aliquoted and stored at −80° C. until use for measurement of analytes.

Serum concentrations of corticosterone, glucagon, and insulin were simultaneously determined by using a magnetic bead assay. All procedures were performed according to manufacturer's instructions, at room temperature and protected from light. Samples were analyzed in a Luminex MAGPIX system. Analyte concentrations were calculated using Analyst software with a five-parameter logistic curve-fitting method.

Western Blot Analysis

Brain segments in the right hemisphere (cerebellum for SIRT-1 analysis; hippocampus for BDNF analysis) were sonicated in PBS containing Pierce Protease Inhibitor (Cat #88665), assayed for total protein concentration, and then mixed with SDS sample buffer. The resulting samples were resolved by SDS-PAGE (8% polyacrylamide for SIRT-1 analysis; 16% polyacrylamide for BDNF analysis) and transferred onto PVDF membranes. The antibodies used were: anti-rabbit IgG (Cat #NA 934-1 ml; 1:5000 dilution), mouse anti-beta tubulin (Cat #E7), IRDye 800cw donkey anti-rabbit (Cat #32212; 1:10,000 dilution). For BDNF analysis the primary antibody used was rabbit anti-BDNF N-20 (Cat #SC-546; 1:1000 dilution). For SIRT-1 analysis the primary antibody used was rabbit SIRT-1 (Cat #07-131; 1:500 dilution). The immunoreactive bands were detected using a detection reagent according to the manufacturer's instructions. Bands were analyzed with image analysis software.

Statistical Analysis

Data analysis was performed using IBM SPSS Statistics Software (V21) and GraphPad Prism (V6.0). Specific statistical tests utilized are noted accordingly. Appropriate post-hoc tests were used. Data are presented as mean±standard deviation unless otherwise noted. *, p<0.05; **, p<0.01, were considered significant.

Results

CR Induces Normoglycemia and Inhibits Stress-Induced Hyperglycemia

To evaluate the effect of 14-hrs of CR on glycemia, a contributable outcome factor in countless models of brain injury, arterial blood glucose levels were measured 10 min prior to and after CA. As shown in FIG. 1, the CR group exhibited significantly lower blood glucose levels prior to CA (mean±standard deviation [SD], 142.3±12.8 mg/dL; n=14) in comparison to control (219.5±16.4 mg/dL; n=12; p<0.01). Blood glucose levels remained stable after cardiac arrest in the CR group (141.8±42.8 mg/dL) but not in the control group (277.7±30.6 mg/dL), which exhibited a significant increase in glucose levels compared to pre-CA control values (p<0.01). These results suggest that rats undergoing asphyxial CA display a stress-induced hyperglycemic response that is inhibited in entirety by 14-hrs of caloric restriction.

CR Improves Survival after Cardiac Arrest

Given the overall severity of the CA model attributable to an 8-min duration of asphyxia, several mortalities were expected. In the control group, two rats failed to resuscitate and an additional two deceased at 24 h and 48 h post-ROSC. Remarkably however, all rats in the CR group successfully resuscitated and survived the full term of experimentation (72 h). To assess whether such an acute duration of caloric restriction affects survivability, a Kaplan-Meier survival analysis was conducted. There was a statistically significant difference in survival distributions for the CON versus CR group (p<0.05; FIG. 2). This suggests that 14-hrs of caloric restriction may have significant downstream cardioprotective and neuroprotective effects against ischemic injury.

CR Improves Neurological Recovery

To evaluate neurological recovery post-CA of rats that successfully resuscitated and survived, NDS scores were measured at 4, 24, 48, and 72 h post-ROSC. As shown in Table 1, NDS testing assesses arousal, brainstem reflexes, basic motor strength, gait, and primitive behaviors. In a pre-experimental, healthy state, rats have perfect NDS scores of 70, whereas post-CA all rats exhibit deficits in NDS.

As shown in FIG. 3, the CR group exhibited significantly higher NDS scores in comparison to the control group at every assessed timepoint post-CA. Analysis by two-way repeated measures ANOVA revealed an overall group difference (p<0.05), and significant differences between the control and CR group at 4 h (16.1±5.0 [control n=12] versus 21.7±4.7 [CR n=14]; p<0.05), at 24 h (49.2±9.3 [control n=11] versus 56.9±6.9 [CR n=14]; p<0.05), at 48 h (50.3±12.4 [control n=10] versus 64.2±3.9 [CR n=14]; p<0.05), and at 72 h (52.4±12.5 [control n=10] versus 67.2±2.5 [CR n=14]; p<0.05), by post-hoc t-tests. These results further suggest that a neuroprotective mechanism against ischemic insult may be switched-on as a downstream consequence of 14-hrs of caloric restriction, which in turn augments neurological recovery.

CR Hastens Recovery of Quantitative ECoG as Measured by Burst Suppression Ratio after Cardiac Arrest and Resuscitation

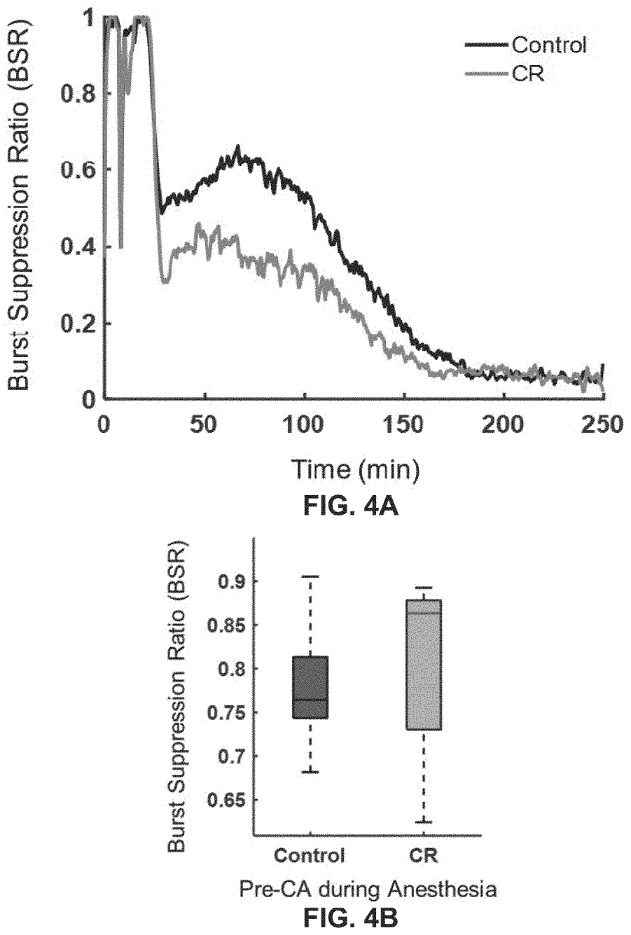

Burst suppression ratio (BSR), a quantitative EEG feature, was used to characterize the amount of suppression present during anesthesia and during early recovery post-CA. Following asphyxia, the CR group had a lower mean BSR, indicating that these rats spent more time in bursting than suppression when compared to control rats. As shown in FIG. 4, the BSR following CA had a significant time-group interaction as revealed by repeated measures ANOVA (p<0.01). While burst suppression is generally associated with poor prognosis after cardiac arrest, it has been reported previously that early increased bursting in rats after asphyxial cardiac arrest is linked to a good outcome. Also, a study in humans reported that a faster recovery of EEG from a bursting pattern to a continuous pattern after cardiac arrest can improve prognostication of a good neurological outcome. Yet another study in humans showed patients with good outcomes had a significantly lower BSR during the first 48 hrs after cardiac arrest than patients with poor outcomes. These results revealed a similar pattern in the BSR when comparing the CR and control groups during the first four hours after CA. Interestingly, during baseline recordings prior to cardiac arrest induction, while under 2% isoflurane anesthesia, there was no significant difference in anesthesia-induced BSR between groups, suggesting that both groups achieved a similar depth of anesthesia, regardless of the ultra-short 14-hr caloric restriction, and hence a similar sensitivity of the brain to isoflurane in CR and non-CR animals.

CR Reduces Neurodegeneration in Multiple Brain Regions

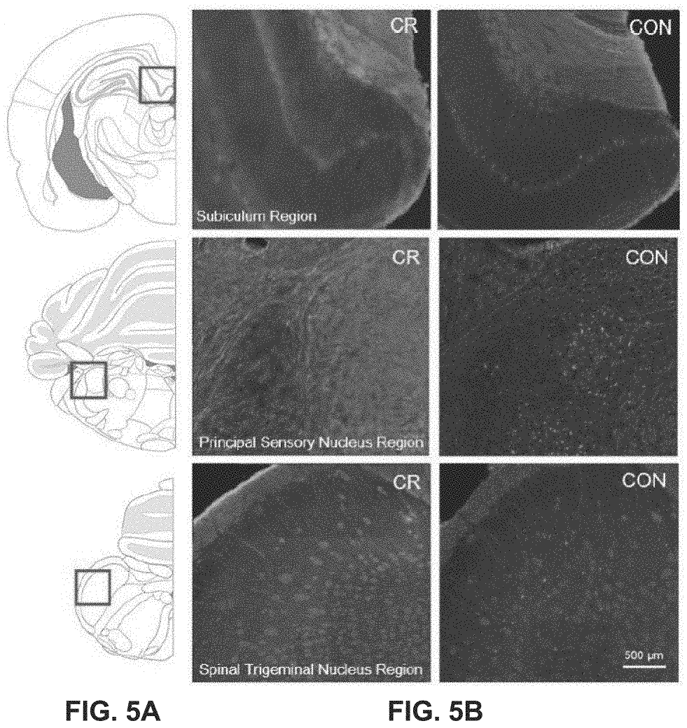

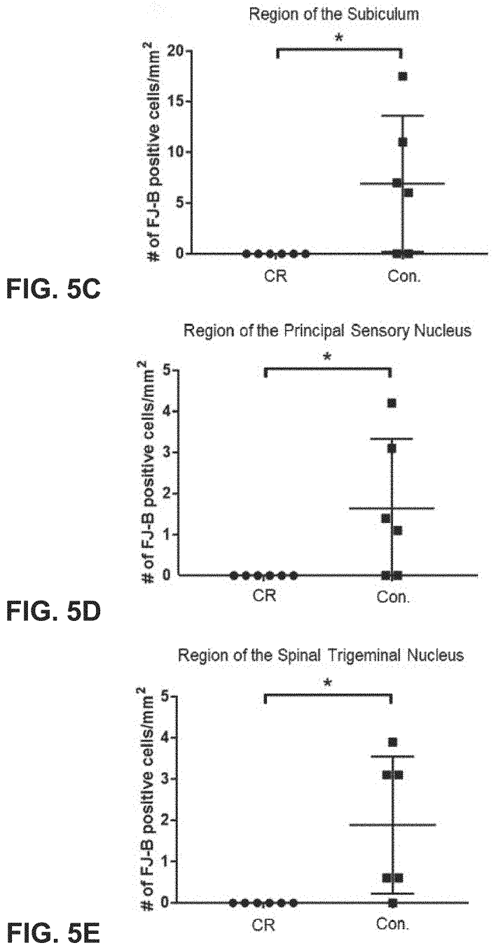

To examine the neuroprotective capacity of overnight caloric restriction at the cellular level, Fluorojade-B (FJ-B) staining was utilized to scan and identify variance in neuronal degeneration between the control and CR group at 72 h post-CA. Images of the areas with FJ-B positive neurons were captured (as indicated by the red squares in FIG. 5A and the representative images for each region in FIG. 5B). Demonstrative of global cerebral ischemia, several physiological regions throughout the brain were identified to have FJ-B positive neurons. All regions were quantified, and three locales were statistically determined to have significant difference between groups. Remarkably, at 72 h post-CA, these regions showed zero FJ-B positive neurons in the CR group (n=6). Comparatively, in the control group FJ-B positive neurons were prevalent in a region of the subiculum (7.3±6.7 cells/mm² [n=6]; p<0.05), a region of the principal sensory nucleus (1.7±1.6 cells/mm² [n=6]; p<0.05), and a region of the spinal trigeminal nucleus (1.9±1.6 cells/mm² [n=6]; p<0.05). These results suggest that 14-hrs of caloric restriction prior to ischemic insult leads to complete neuroprotective effects in certain physiological locales of the brain.

CR Leads to Ketosis

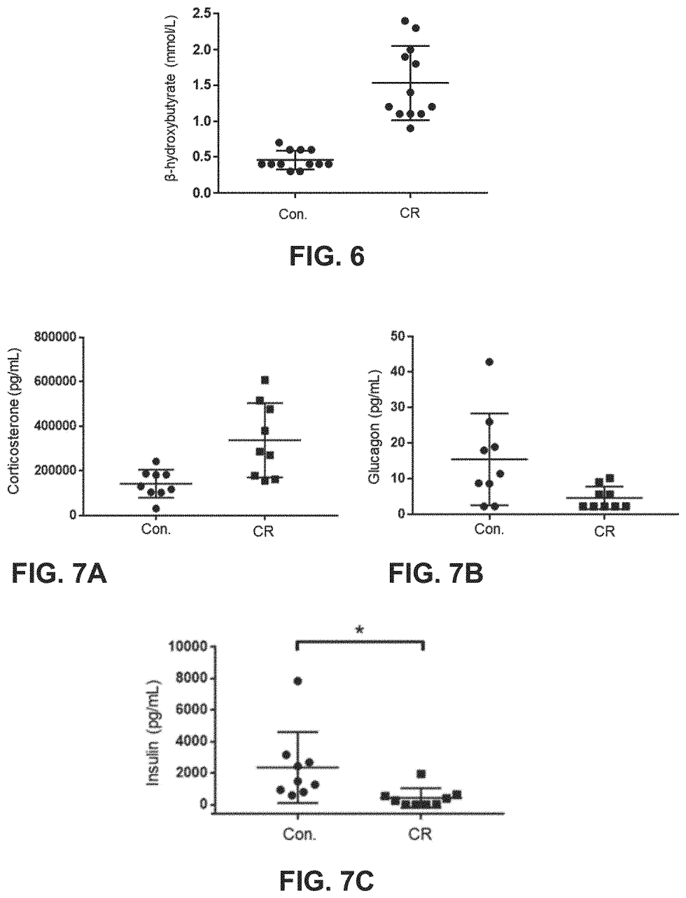

Given that both glycemia and caloric restriction were implicated in ketone body production, capillary blood ketone levels (β-hydroxybutyrate) were measured after 14-hrs of caloric restriction. As shown in FIG. 6, the CR group exhibited significantly higher blood ketone levels (mean±standard deviation [SD], 1.4±0.5 mmol/L; n=12) in comparison to control (0.6±0.2 mmol/L; n=12; p<0.01). These results indicate that 14-hrs of caloric restriction is of sufficient length to significantly increase the production of endogenous blood ketone levels.

CR Leads to Higher Corticosterone and Lower Glucagon and Insulin

As an indicator of stress, and to further elucidate upon the potential role of glycemia as a contributable outcome factor, corticosterone, glucagon, and insulin levels were assessed in arterial blood collected after 14-hrs of caloric restriction. As shown in FIG. 7A-7C, the CR group exhibited significantly higher corticosterone levels in comparison to the control group ([n=12]; p<0.05), likely indicative of a stress-response following metabolic deficits. Blood levels of glucagon, on the contrary, were significantly lower in the CR group in comparison to the control group ([n=9]; p<0.05). Blood levels of insulin were likewise significantly lower in the CR group in comparison to the control group ([n=8]; p<0.05).

CR does not Change Expression of SIRT1 and BDNF

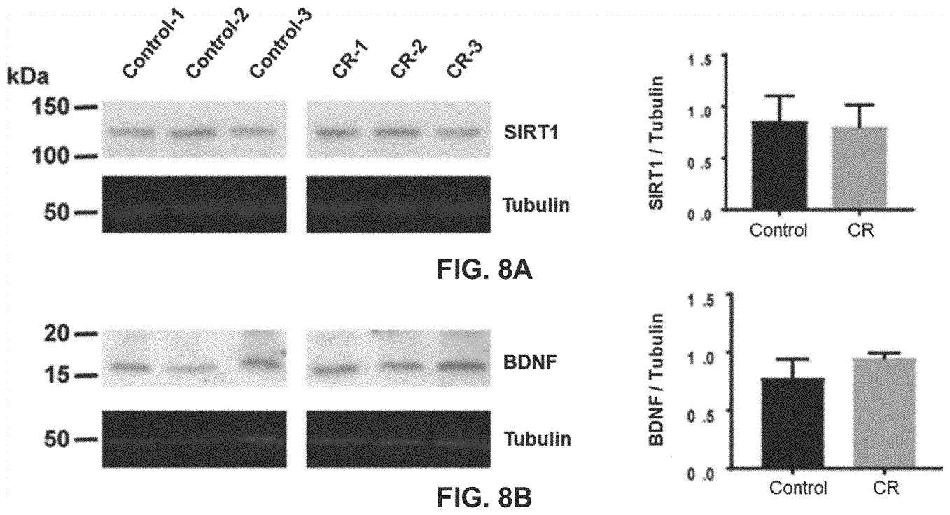

Caloric restriction is known to upregulate brain-derived neurotrophic factor (BDNF) and sirtuin 1 (SIRT1) pathways in the brain, particularly following subacute periods of dietary restriction. To assess the potential upregulation of 14-hrs of caloric restriction, Western blot analyses were conducted on brain homogenates of a separate cohort of rats that were calorically restricted for 14-hrs. Surprisingly, as shown in FIG. 8, no significant differences were found in either BDNF or SIRT-1 expression after 14-hrs of caloric restriction in comparison to controls.

Without wishing to bind the invention to a particular theory, these results suggest that a 14-h period of overnight caloric restriction prior to cardiac arrest and resuscitation in a rodent model has significant effects on survivability and neurological recovery.

### Example 2: Generation of Neuroprotective Nutrition Program

A 67-year-old male patient presents to the emergency department complaining of chest pains. The attending physician examines the patient by conducting a physical exam, including auscultation while checking his vital signs, electrocardiogram, chest x-ray, bedside echocardiogram, and blood tests results that include cardiac enzymes in addition to standard blood tests and any prior medical records suggestive of coronary artery disease and potential cardiac ischemia. Based on the results of the examination, the physician determines that the patient is at high risk of cardiac arrest. The physician evaluates the cerebral metabolic state of the patient by using a portable optical device which measures CBF and CMRO₂. Because the CBF/CMRO₂ ratio is 0.5 (which is significantly less than 1), the physician determines that a neuroprotective nutrition program for the patient should include acute caloric restriction of 75% for a period of 20 hours. The physician generates a neuroprotective nutrition program for the patient which includes a caloric limit of 500 kilocalories for the next 20 hours. At the end of the 20-hour period, the physician re-evaluates the cerebral metabolic state of the patient and determines that the CBF/CMRO₂ ratio has increased to 0.9. Based on this change, the physician generates a revised neuroprotective nutrition program for the patient which includes a daily caloric limit of 1500 kilocalories for the next 72 hours. The patient makes dietary decisions based on the revised neuroprotective nutrition program.

### Example 3: Inducement of Early Spreading Depolarization Via Ketone Injection

A 55-year-old female patient presents to the emergency department complaining of chest pains. While in the hospital, the patient experiences cardiac arrest. During the cardiac arrest, the patient's cerebral metabolic state is monitored via a portable optical/EEG device. During entry into cardiac arrest, it is seen that the patient's brain metabolism (measured by absolute CMRO₂) is low (e.g., <3 ml O₂/min/100 g). Therefore, this patient is determined to be at risk of delayed spreading depolarization, potentially leading to worse neurological outcome. Based on this real-time feedback, ketone injection of beta-hydroxybutyrate (1.5 gm/kg body weight), followed by a continuous infusion of beta-hydroxybutyrate (0.18 gm/kg/hour), is given to mimic a calorically-restricted state and induce spreading depolarization earlier so as to provide neuroprotection during cardiac arrest.

### Example 4: Iterative Generation of a Neuroprotective Nutrition Program Via a Wearable Device

A 48-year-old male patient with a history of coronary artery disease presents to his primary care physician with shortness of breath and a rapid and irregular heartbeat. The physician prescribes use of a wearable device to generate a neuroprotective nutrition program for the patient based on monitoring of the patient's metabolic state. The patient wears the wrist-mounted device for 12 hours a day and receives hourly automatic updates to a digital nutrition program on his phone based on his metabolic rates of glucose and oxygen consumption. This device uses a multivariate linear regression algorithm (see [0028]) to make updated personalized calculations of the patient's recommended percentage of caloric restriction each day, based on the patient's metabolic rates of glucose and oxygen consumption and other risk factors. The patient makes dietary decisions based on the digital nutrition program.

### Example 6: Applications to Hypoxic-Ischemic States Originating from Non-Cardiac Sources that can Lead to Cardiac Arrest

A 70-year old female is admitted with shortness of breath. She has no history of prior medical conditions and just returned from a long overseas trip. On workup in the emergency room, she is found to be hypoxic with pulse oximeter around 90% oxygen saturation and tachycardic. CT angiography of the lungs demonstrate a pulmonary embolism and the source is found to be a deep venous thrombosis in her right leg. She is started on intravenous anticoagulation medications and admitted to the intensive care unit, later becoming more hypoxic requiring intubation and mechanical ventilation. She begins needing higher ventilator support while also becoming hypotensive requiring vasopressors. Because she is now at a high risk of a cardiac arrest caused by the pulmonary embolism and its consequent hypoxia and hypotension, her cerebral and global metabolic state is assessed using a bedside portable device measuring CBF and CMRO₂. Since her CBF/CMRO2 ratio is found to be suboptimal at 0.7, the medical team optimizes her nutrition to maximize neuroprotection and cardioprotection. Acute caloric restriction is instated at 90% for the next 24 hours to limit her calorie intake to 200 kilocalories, and this takes into account both enteral nutrition through a nasogastric tube as well as dextrose infusion through her intravenous catheters. To optimize ketosis, a majority of these calories are derived from proteins and lipids rather than carbohydrates. At the end of the 24-hour period, her urinalysis shows that her body is producing and excreting ketones. Her serum beta-hydroxybutyrate levels corroborate the urine sample as they are also significantly higher. The intensive care unit team which includes the physicians in conjunction with the nutritionists determines that her CBF/CMRO₂ ratio has increased to 0.95. Her oxygen saturation and blood pressure have slightly stabilized, but she is still critically ill. It is determined that for the next 24-hour period, her caloric limit will be raised to 500 kilocalories to maintain 75% caloric restriction. Over the next 24-hour period, she continues to improve, and her risk of a cardiac arrest is significantly lower. She is on minimal ventilator settings and off of vasopressors. The medical team finds that her CBF/CMRO₂ ratio has maintained itself around 0.9 while still in a mild ketosis state based on her lab tests. Since she is continuing to improve, her calorie restriction is lowered again so that she will only require 50% calorie reduction over the following day. Daily or more frequent assessments are done to continually adjust the caloric intake regimen for the patient's needs.

The examples above demonstrate risk factors and causes of potential cardiac arrest that may be cardiac or non-cardiac in origin. As the above examples demonstrate, global ischemia to the body, including the brain, can be mild, moderate, or severe, the latter of which constitutes a state of circulatory shock that can include cardiac arrest. Causes of worsening global ischemia and severe shock can be cardiac or non-cardiac in origin. Cardiac causes can include myocardial infarction caused by coronary artery disease, cardiac arrhythmia caused by genetic predisposition or environmental insults. Pulmonary causes can be due to hypoxia resulting from massive pulmonary embolism, acute respiratory distress syndrome (e.g. ARDS), severe pneumonia due to infection or aspiration, severe pulmonary edema, or other causes. Hypercoagulable states and inflammatory states can have a major impact on these causes, and this can include COVID-19 related causes that can frequently cause hypoxia or hypercoagulability that can lead to cardiac arrest. Moreover, circulatory shock can be due to severe sepsis (e.g. septic shock), cardiac shock not necessary caused by acute coronary syndrome (e.g. severe heart failure such as Takotsubo disease), hemorrhagic shock (e.g. trauma or related incident causing internal or external haemorrhage), neurogenic shock (e.g. impending brain death due to acute brain injury leading to severe cerebral edema and brain herniation), spinal shock (e.g. due trauma or other cause), hypovolemic shock (e.g. due to severe dehydration), or other types of conditions alone or in a combination thereof of any of these conditions that can lead to a state of hypoxia and/or ischemia to specific regions of the body, including or excluding the brain. Thus, the risk of cardiac arrest may stem from a risk of any of the conditions above and the means for evaluating risk of cardiac arrest may include a means for evaluating any of the conditions above.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

What is claimed is:

1. A system for generating a neuroprotective nutrition program for a patient, the system comprising:
a. a means for evaluating the patient's risk of cardiac arrest; and
b. a means for evaluating a metabolic state of the patient to determine the neuroprotective potential of caloric restriction of the patient;
wherein combination of the patient's risk of cardiac arrest and neuroprotective potential of caloric restriction allow for generation of the neuroprotective nutrition program.

2. The system of claim 1, wherein the means for evaluating the metabolic state of the patient is configured to evaluate a cerebral metabolic state, a non-cerebral metabolic state, or a combination thereof.

3. The system of claim 2, wherein the system is configured to allow for comparison of a cerebral metabolic state and a non-cerebral metabolic state so as to assess autoregulation.

4. The system of claim 1, wherein the neuroprotective nutrition program comprises a neuroprotective dietary limit.

5. The system of claim 1, wherein the neuroprotective nutrition program is for a period of less than 72 hours.

6. The system of claim 1, wherein the neuroprotective nutrition program is for a period of less than 24 hours.

7. The system of claim 1, wherein the neuroprotective nutrition program includes a set ratio or amount of macronutrients.

8. The system of claim 1, wherein the means for evaluating the patient's risk of cardiac arrest comprises a means for evaluating clinical signs and symptoms, an oxygen saturation monitor, a pulse oximeter, a blood pressure monitor, heart rate monitor, a heart rhythm monitor, an electrocardiogram device, a serum laboratory profile, a serum laboratory profile including cardiac enzymes, a coronary catherization or angiogram device, a chest x-ray device, an echocardiogram device, a computed tomography (CT) device, a magnetic resonance imaging (MRI) device, a diffuse optical spectroscopy device, a near infrared spectroscopy (NIRS) device, a perfusion monitoring device, a laser Doppler flowmetry device, a nuclear scan device, a genetic test, or a combination thereof.

9. The system of claim 1, wherein the means for evaluating a metabolic state of the patient is configured to measure cerebral metabolic rate of oxygen (CMRO₂), cerebral metabolic rate of glucose, cerebral blood flow (CBF), flow-metabolism ratio (CBF/CMRO₂) tissue concentration of deoxy-hemoglobin (ctHb), tissue concentration of oxygenated hemoglobin (ctHbO₂), tissue oxygenation (StO₂), tissue water content, tissue lipid content, tissue reduced scattering coefficient, tissue scattering amplitude, tissue scattering slope, tissue reflectance, or a combination thereof.

10. The system of claim 1, wherein the patient's metabolic state is evaluated based on most recent meal times, glucose levels, corticosterone levels, glucagon levels, insulin levels, ketone levels, or a combination thereof.

11. The system of claim 1, wherein the means for evaluating the patient's metabolic state is configured to measure one or more analytes from a biological sample taken from the patient.

12. The system of claim 11, wherein the biological sample is a blood, tissue, saliva, sweat, cerebral spinal fluid, respiratory gas, or urine sample.

13. The system of claim 11, wherein the biological sample is evaluated using techniques including, but not limited to, metabolomics, proteomics, transcriptomics, and other related technologies, to obtain data on the concentrations of endogenous metabolites in the sample and quantify how these metabolite concentrations change as a result of caloric restriction

14. The system of claim 1, wherein the means for evaluating the patient's cerebral metabolic state comprises a wearable device.

15. A system for evaluating the neuroprotective potential of caloric restriction of a patient, the system comprising:
a. a means for evaluating oxygen or glucose supply to the patient's brain; and
b. a means for evaluating the patient's cerebral metabolic rate of oxygen or glucose;
wherein the combination of data on oxygen or glucose supply and cerebral metabolic rate allows for evaluation of the neuroprotective potential of caloric restriction of the patient.

16. The system of claim 14, wherein the system additionally comprises a means for evaluating a non-cerebral metabolic rate of oxygen or glucose.

17. A method for generating a neuroprotective nutrition program for a patient, the method comprising:
a. evaluating the patient's risk of cardiac arrest;
b. evaluating the patient's metabolic state; and
c. generating a neuroprotective nutrition program for the patient based on combination of the patient's risk of cardiac arrest and metabolic state.

18. The method of claim 17, wherein the method comprises re-evaluating the patient's metabolic state after a time period and amending the neuroprotective nutrition program based on a change in the cerebral metabolic state.

19. The method of claim 17, wherein the method comprises collection, isolation, and preservation of blood serum from a patient during a calorically restricted state and re-administration of that blood serum to the patient during a time period when a patient is not calorically restricted, in order to confer neuroprotection

20. The method of claim 17, wherein the method comprises synthesis of a cocktail of one or more metabolites known to increase in concentration during caloric restriction, and administration of that cocktail to the patient during a time period when they are not calorically restricted, in order to confer neuroprotection

21. A system for improving neurological outcome in a patient during cardiac arrest, the system comprising:
a. a means for evaluating the patient's cerebral metabolic state; and
b. a means for inducing spreading depolarization of the patient;
wherein the system allows for inducement of spreading depolarization of the patient at a specific time during or after cardiac arrest, based on the patient's cerebral metabolic state.

22. The system of claim 21, wherein the means for inducing spreading depolarization comprises a therapeutic configured to place the patient in a state that mimics a caloric restricted state, a therapeutic treatment configured to increase ketone levels, a therapeutic treatment configured to lower glucagon and insulin levels, chemical stimulation, physical stimulation, electrical stimulation, magnetic stimulation, optical stimulation, soundwave-induced stimulation, or a combination thereof.

23. The system of claim 21, wherein the means for inducing spreading depolarization comprises imposing caloric restrictions on the patient prior to the cardiac arrest.

24. The system of claim 21, wherein the system is configured to induce spreading depolarization at an earlier time or a later time depending on the patient's cerebral metabolic state.

25. The system of claim 21, wherein the system additionally comprises a means for inducing repolarization in the patient at a specific time after resuscitation, reinstatement of perfusion, or reinstatement of oxygenation, wherein said time depends on the metabolic state of the patient.

26. The system of claim 25, wherein the means for inducing repolarization is configured to modify the patient's blood pressure, cerebral blood flow, cerebral metabolism, or a combination thereof, via chemical stimulation, physical stimulation, electrical stimulation, magnetic stimulation, optical stimulation, soundwave-induced stimulation, or a combination thereof.

27. A system for generating a short-term cardioprotective nutrition program for a patient, the system comprising:
a. a means for evaluating the patient's risk of cardiac arrest; and
b. a means for evaluating a metabolic state of the patient to determine the neuroprotective potential of caloric restriction of the patient;
wherein combination of the patient's risk of cardiac arrest and cardioprotective potential of caloric restriction allow for generation of the cardioprotective nutrition program.

28. A system for evaluating the cardioprotective potential of caloric restriction of a patient, the system comprising:
a. a means for evaluating oxygen or glucose supply to the patient's brain; and
b. a means for evaluating the patient's cerebral metabolic rate of oxygen or glucose;
wherein the combination of data on oxygen or glucose supply and cerebral metabolic rate allows for evaluation of the cardioprotective potential of caloric restriction of the patient.

29. The system of claim 28, wherein the biological sample is evaluated using techniques including, but not limited to, metabolomics, proteomics, transcriptomics, and other related technologies, to obtain data on the concentrations of endogenous metabolites in the sample and quantify how these metabolite concentrations change as a result of caloric restriction

30. A method for generating a cardioprotective nutrition program for a patient, the method comprising:
a. evaluating the patient's risk of cardiac arrest;
b. evaluating the patient's metabolic state; and
c. generating a cardioprotective nutrition program for the patient based on combination of the patient's risk of cardiac arrest and metabolic state.

31. The method of claim 29, wherein the cardioprotective nutrition program is for a period of less than 72 hours.

32. The method of claim 29, wherein the cardioprotective nutrition program comprises collection, isolation, and preservation of blood serum from a patient during a calorically restricted state and re-administration of that blood serum to the patient during a time period when a patient is not calorically restricted, in order to confer cardioprotection

33. The method of claim 29, wherein the cardioprotective nutrition program comprises synthesis of a cocktail of one or more metabolites known to increase in concentration during caloric restriction, and administration of that cocktail to the patient during a time period when they are not calorically restricted, in order to confer cardioprotection.
